(19) United States
(12) Patent Application Publication
(10) Pub. No.: US 2022/0409828 A1
(43) Pub. Date: Dec. 29, 2022

(54) NOZZLE WITH FAN FOR DELIVERING AEROSOLS

(71) Applicant: BOA Nutrition, Inc., Raleigh, NC (US)

(72) Inventors: Hank Durschlag, Raleigh, NC (US); Jon Pritchett, Raleigh, NC (US)

(21) Appl. No.: 17/838,773

(22) Filed: Jun. 13, 2022

Related U.S. Application Data
(60) Provisional application No. 63/209,748, filed on Jun. 11, 2021.

(51) Int. Cl. A61M 11/00

(52) U.S. Cl. CPC A61M 11/006; A61M 11/001; A61M 2210/0625

(57) ABSTRACT

A system for delivering a composition containing a nutritional supplement matrix to a user through the oral mucosa, in which the matrix is held under pressure in a canister that has a nozzle with at least one rotating internal member positioned between the canister outlet and the nozzle outlet, in which the rotating member agitates the composition to increase atomization to aid in absorption.

The present application claims priority to U.S. Application 63/209,748 filed Jun. 11, 2021, which is incorporated by reference.

### FIELD OF THE INVENTION

The present invention is generally related to nutritional supplements' delivery through the oral mucosa, specifically the nozzles needed to deliver them.

### BACKGROUND

Pump spray bottles for oral delivery of pharmaceuticals and other liquids are well known. While many advances have been made in dosing accuracy for this type of delivery method, there has not been a focus on “ease of use” while engaging in physical activity. The “ease of use” is related to the relative scarcity of aerosol applications. They are for absorption through the buccal mucosa. Those applications, such as insulin or methadone, are not frequently given to someone engaged in an athletic activity. Buccal adhesive patches are an alternative delivery route that can be maintained during athletic training and pose difficulties in administration while active. An easily directable, powerful, atomized, aerosol spray of an electrolyte composition with both a mucoadhesive, such as pectin and an absorption enhancer, such as a liposomal delivery system, is ideal for replacing lost electrolytes during strenuous exercise.

Compressed gas, such as oxygen, offers a few advantages as an aerosol propellant. They do not burn how another liquid aerosol propellant can, and they are readily available and inexpensive. Oxygen compressed gas aerosol propellants are not used in modern aerosols as the pressure can reduce over the lifetime of the aerosol. The lifetime issue is partly because the single volume inside the can increases as the product is used up, and some of the propellants are released. When the compressed gas is a desired deliverable portion of the product, it becomes essential to overcome these disadvantages. While over pressurizing the can does mitigate this issue somewhat, an additional mechanism that can aid the propellant in ejecting the product with sufficient force, atomizing, and direction would be desirable.

The buccal mucosa has low enzymatic activity that can interfere with absorption. It offers a large, vascular, absorptive area with excellent systemic circulation access through the internal jugular vein. This path also allows products to bypass first-pass metabolism, which may increase the product's bioavailability. The relative immobility of the buccal mucosa may also help increase the residence time of drugs. The tongue, hard palate, and laryngeal mucosa do not offer all of these benefits to deliver actives to the bloodstream. Therefore, it is desirable to have a very directable spray pattern for applying actives to the buccal mucosa.

Mucoadhesives, such as apple pectin, are an excellent way to increase the residence time of drugs on mucosal surfaces. To that end, several mucoadhesive films are known in the art for delivering drugs through the buccal mucosa to benefit from the enhanced bioavailability that route provides. Pectin, and other mucoadhesives, tend to agglomerate, making them difficult to apply in spray applications due to irregular atomization of the spray and potentially clogging of the nozzle. It is, therefore, desirable to have a mechanism to mechanically break up agglomerated particles.

In many spray applications, it is the propellant responsible for providing sufficient pressure to deliver an accurate dose throughout the life of the can, properly atomize the product being sprayed, prevent aggregation and clogging, and allow the product to be accurately directed to the appropriate absorption point. In applications in which the propellant is a compressed gas, it is beneficial to have a mechanism that will assist the propellant with at least one. Helping the propellant is particularly important when the compressed gas is a part of the deliverable product, making the loss of pressure more pronounced at the end of life.

### BRIEF DESCRIPTIONS OF THE DRAWINGS

FIG. 1 illustrates a nozzle housing with a rotating member.

FIG. 2 illustrates the nozzle housing with the rotating member engaged to a canister.

FIG. 3 illustrates a nozzle housing with a small rotating member.

FIG. 4 illustrates a nozzle housing with dual rotating members.

FIG. 5A illustrates a nozzle housing with a rotating member positioned after the nozzle.

FIG. 5B illustrates the nozzle housing with the rotating member positioned after the nozzle.

### SUMMARY

Systems for delivering an aerosol composition to an oral mucosa are described. A nozzle is connected or engaged to a supply of the aerosol composition under pressure. When the systems emit the aerosol composition, rotating member or fans agitate the aerosol to increase atomization and/or reduce agglomeration. The systems may direct the aerosol composition through blades or other rotating members. The release of the aerosol composition, under pressure, may drive the rotating movement of the rotating members or the fans.

In one aspect, a system for delivering a composition to an oral mucosa is described. The system includes a canister containing pressurized air and at least one liquid composition. The system includes a nozzle housing having a lower opening and an outlet. The lower opening and the outlet are connected by a flow path. The lower opening of the nozzle housing is engaged to the canister to receive an aerosol mixture of the at least one liquid composition from the canister. The outlet is configured to emit the aerosol mixture of the at least one liquid composition. The nozzle housing positions at least one rotating member in or after the flow path.

In another aspect, a nozzle housing for emitting pressurized air and at least one liquid composition is described. The nozzle housing includes a lower opening and an outlet. The lower opening and the outlet connected by a flow path. The lower opening of the nozzle housing is configured to engage to a supply of a pressurized air and at least one liquid composition. The outlet is configured to emit an aerosol mixture of the at least one liquid composition. The nozzle housing positions at least one rotating member in or after the flow path. The at least one rotating member is configured to agitate the aerosol mixture of the at least one liquid composition.

In another aspect, a method of delivering an aerosol mixture of oxygen and at least one liquid composition is described. The method includes providing a nozzle housing having a lower opening and an outlet. The lower opening and the outlet are connected by a flow path. The method further includes directing an aerosol mixture of pressurized gas and at least one liquid composition through the flow path of the nozzle housing. The method further includes positioning at least one rotating member in or after the flow path. The method further includes causing the rotating member to rotate by the aerosol mixture. The method further includes agitating the aerosol mixture and delivering the aerosol mixture to an oral mucosa.

### DETAILED DESCRIPTION

For purposes of this application, any terms that describe relative position (e.g., “upper”, “middle”, “lower”, “outer”, “inner”, “above”, “below”, “bottom”, “top”, etc.) refer to an aspect of the invention as illustrated, but those terms do not limit the orientation in which the aspect can be used.

Transmucosal delivery of nutrient supplements offers advantages over oral delivery when negative issues relating to the gastrointestinal tract, stomach, substance digestion and absorption, swallowing, protocol compliance, substance effectiveness, and other gastrointestinal metabolism problems are considered. The formulation or composition may include a nutritional supplement matrix fraction, (ii) a gas fraction, (iii) an enhancer fraction, (iv) a liquid fraction, and (v) a preservative fraction wherein the nutritional supplement matrix fraction, the gas fraction, the enhancer fraction, the liquid fraction, and the preservation fraction are all mixed or combined and treated to maintain a state of balanced suspension among the oxygen molecules for a specific duration of time before being dispensed from a canister. Furthermore, the composition may be in a compressed state in a canister before being dispensed. In another aspect, the formulation or composition would include a nutritional supplement matrix fraction, (ii) a gas fraction, (iii) an enhancer fraction, (iv) a liquid fraction, and (v) a preservative fraction wherein the nutritional supplement matrix fraction, the enhancer fraction, the liquid fraction, and the preservation fraction are all mixed or combined and stored separately from the gas fraction in a canister before being dispensed. Additionally, the nutritional supplements are formulated, treated, and mixed with the gas fraction. The gas fraction is oxygen to maintain a state of balanced suspension among the oxygen molecules for a specific duration of time after being dispensed.

Furthermore, the composition may be in a compressed state in a canister before being dispensed. The transmucosal delivery of the supplement matrix is more efficient when the composition is atomized. It allows the enhancer fraction of the composition to adhere more to the nutritional supplement to the mouth's mucosal membranes. The buccal mucosa offers a promising administration site for nutrients as it has a rich blood supply and is relatively permeable. The bioadhesion of the delivery system is crucial for delivering across the buccal mucosa. Saliva may wash the delivery method of the buccal region. To alleviate these hydrophobic patches have been used to hold the nutrient or medication being administered through the buccal mucosa. Buccal spray devices have been used to deliver insulin in a mist of fine droplets onto the mucin layer of the mucosal membrane. Without a hydrophobic coating to protect the desired deliverable from the saliva, it is crucial to atomize the mixture and deliver it at a high velocity directly onto the buccal mucosa to maximize the amount of deliverable that is absorbed.

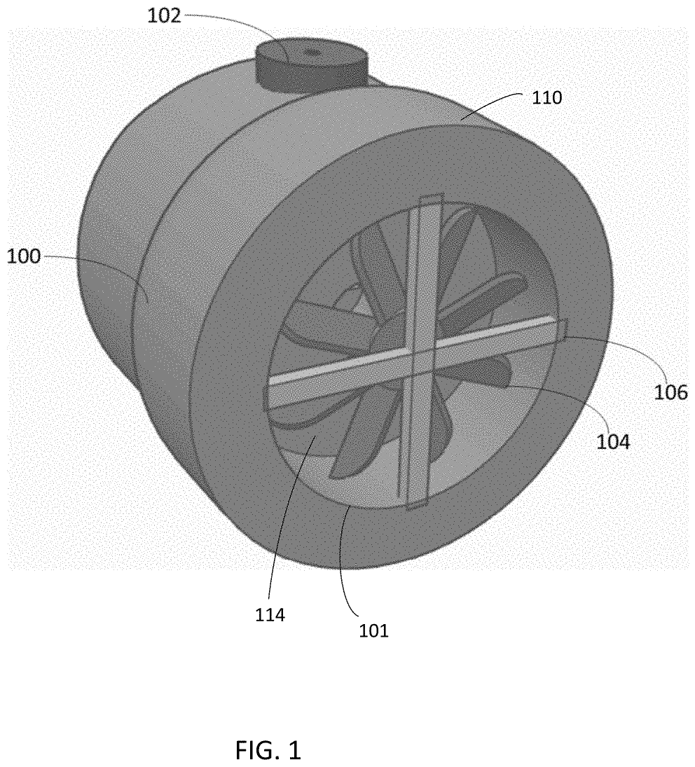

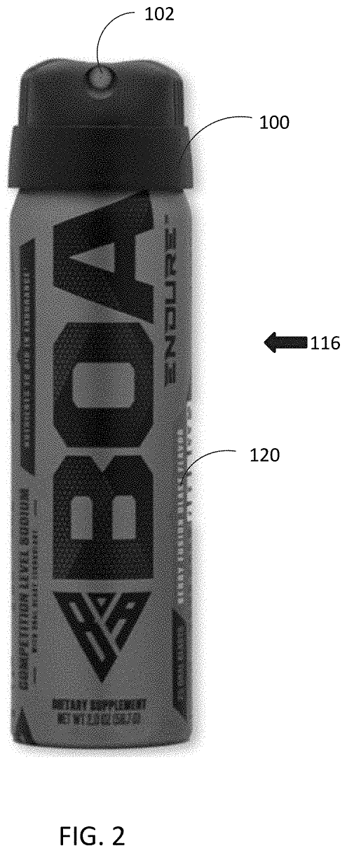

FIG. 1 is a perspective view of a first nozzle housing 100 for delivering an atomized mixture of oxygen and nutritional supplements into a mouth of a user for transmucosal delivery. FIG. 2 illustrates a system for delivering a composition to the oral mucosa 116 with the nozzle housing 100 affixed or engaged to a top of a canister 120 containing the nutritional supplements under pressure. In this aspect, a lower opening 101 of the nozzle housing 100 is engaged to the canister 120. The nutritional supplement passes from the canister 120 to the nozzle housing 100. The lower opening 101 may engage an outlet of the canister 120.

The nozzle housing 100 includes an outlet 102 that allows the atomized mixture or aerosol to exit the nozzle housing 100 and enter the user's mouth. The nozzle housing 100 may include any of many nozzle shapes known in the art that can adjust the spray pattern into the mouth for transmucosal delivery. For example, the nozzle housing 100 may provide a flat fan, full cone, hollow cone, solid stream, etc.

A rotating member 104 is positioned within the nozzle housing 100 between the canister 120 and the outlet 102. The rotating member 104 is affixed to the nozzle housing 100 by a support member 106. In the aspect FIG. 1, the support member 106 is located near a base 110 of the nozzle housing 100, which is close to the canister 120. The support member 106 extends across a width of an interior of the nozzle housing 100. The rotating member 104 is rotatably engaged to the support member 106. In this aspect, the rotating member 104 is affixed to an axle on the support member 106 that allows the rotating member 104 to spin inside of the nozzle housing 100 to agitate the composition being propelled into the mouth. The rotating member 104 may include multiple blades rotating in a lower cavity 114 of the nozzle housing 100. The lower cavity 114 is fluidly connected to the outlet 102 such that the nutritional supplements pass from the lower cavity 114 upward to the outlet 102.

The agitation of the composition by the rotating member 104 will further atomize the composition being propelled into the mouth, allowing for better absorption of the composition through the oral mucosa. When a mucoadhesive, such as apple pectin, is incorporated into the formulation, the droplets will be more prone to agglomeration. Incorporating the rotating member 104 or fan element into the nozzle housing 100 will further break up the agglomerated particles. The rotating member 104 will allow for better atomization of the formulation to increase absorption through the buccal mucosa and prevent the nozzle outlet 102 from clogging with agglomerated particles. The design, angle, and the several blades in the rotating member 104 may be optimized for this purpose. In this aspect, the rotating member 104 has its blades positioned to cause the rotating member 104 to spin when the compressed air is released from the canister. The blades of the rotating member 104 may be angled to ensure the canister's best atomization of the material. In certain aspects, a direction of rotation of the rotating member 104 may be generally perpendicular to a flow-path of the composition.

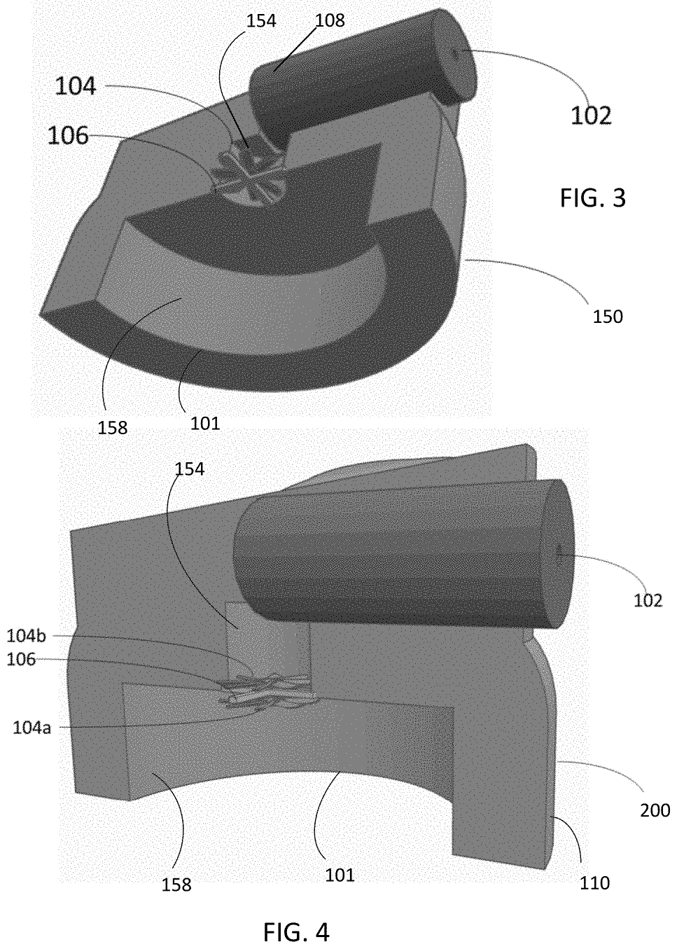

FIG. 3 illustrates a second nozzle housing 150. A cross-section of second nozzle housing 150 shows a smaller variation of the rotating member 104 connected to the support member 106 in an interior 152 of the second nozzle housing 150. The rotating member 104 is located in an upper cavity 154 of the second nozzle housing 150 close to the outlet 102. The upper cavity 154 is over a lower cavity 158 of the second nozzle housing 150.

The upper cavity 154 has a smaller internal diameter than an internal diameter of the lower cavity 158 of the second nozzle housing 150. Thus, the interior 152 of the second nozzle housing 150 tapers inward in the upward direction—in the direction of the outlet 102. A rear portion of an outlet housing is fluidly connected to the upper cavity 154. By positioning the rotating member 104 in the narrower upper cavity 154, the rotating member 104 performs more rotations per minute under the same amount of pressure compared to the aspect illustrated in FIG. 1. Greater RPM may further atomize the composition or achieve a similar RPM to the rotating member in FIG. 1 with a lower amount of air pressure from the canister. Positioning the rotating member 104 in a narrow portion of the nozzle housing 100, i.e., the upper cavity 154, may allow for greater air velocity, with the same canister air pressure, at the point of contact with the rotating member 104 due to the narrowing of the nozzle housing 100.

FIG. 4 illustrates a third nozzle housing 200 having a dual fan aspect. In this aspect, a second rotating member 104b is affixed to the opposite side of the support member 106 with a connection, through the support member 106, to a rotating member 104a. In this aspect, the two rotating members 104a and 104b have the same size and orientation. The second rotating member, 104b in this aspect, serves to further agitate the composition to aid in atomization. The angle and number of fan blades on each of the rotating members 104a and 104b may be adjusted to increase the composition's velocity and atomization. In other aspects, the fans may rotate independently of one another. In still further aspects, the fans may be of differing sizes. Two different size rotating members 104a and 104b may allow for one rotating member 104a to drive the rotation of the second rotating member 104b, allowing the second rotating member to rotate at a speed that exceeds the rate it would achieve based on its shape under the amount of pressure produced by the canister. In still further aspects, one or both of the rotating members 104a and 104b may be actively powered. Power may be supplied by a battery and actuator or through the mechanical movement of the nozzle housing 100 to the canister.

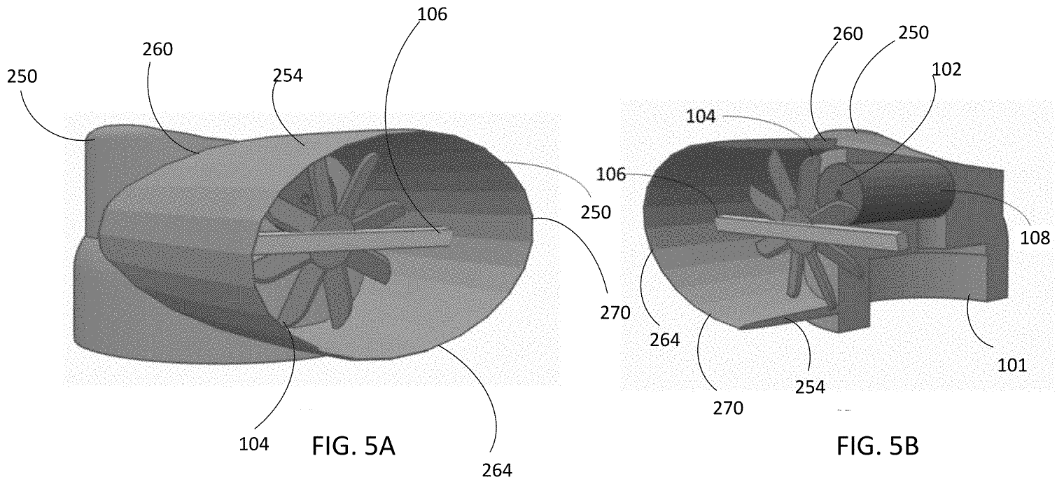

FIGS. 5A and 5B illustrates a fourth nozzle housing 250 in which the rotating member 104 is located after the outlet 102 of the aerosol. The fourth nozzle housing 250 emits the aerosol from the outlet 102, and then then the aerosol passes through the rotating member 104. The fourth nozzle housing 250 may include an additional structure to direct the flow path of the aerosol into the mouth of the user. As shown in FIGS. 5A and 5B, the fourth nozzle housing 250 includes a tubular member 254. The tubular member 254 is positioned over the outlet 102. A portion of the outlet 102 may extend in the tubular member 254. A proximal end 260 of the tubular member 254 is connected or integral with the fourth nozzle housing 250. A distal end 264 of the tubular member 254 includes an opening 270 to permit the flow of the aerosol to the user. In this aspect, the support member 106 may extend across an inner diameter of the tubular member 254. The support member 106 may engage opposites side of an interior of the tubular member 254. The support member 106 positions the rotating member 104 in the flow path of the aerosol. After the aerosol is emitted from the outlet 102, the aerosol passes through the rotating member 104, and then to the user.

In other aspects, the rotating member 104 may be positioned to accelerate the flow of particles into the mouth to increase the penetration into the buccal mucosa. The rotating member 104 may also be oriented to direct the flow of atomized droplets to the mouth's proper regions. For example, the rotating member 104 could be tilted downward to allow the user to control the spray more easily to one side of their mouth or the other and avoid spraying into the back of the throat where the nutritional absorption may be less efficient. To achieve the same benefit in a single spray, a pair of rotating members 104 could be positioned in the nozzle housing 100, each angled out to allow the user to spray directly into the mouth and have the rotating members 104 direct the droplets away from the back of the throat and towards the buccal region.

The nozzle housings described herein may also be used sprays that manually pressurize the liquid composition. For example, pump sprayers or trigger activated sprayers may develop sufficient PSI to dispense an aerosol. The rotating members and fans described herein may be utilized with such sprayers to increase atomization and reduce agglomeration.

The canister 120 may contain a supplement fraction, a gas fraction, an enhancer fraction, a liquid fraction, and a preservation fraction under pressure.

The supplement composition may include a combination of electrolytes, vitamins, and/or minerals. The supplement composition may be selected from or be combination of Potassium chloride, Sodium chloride, Iron, Sodium, Calcium, Magnesium, Carbohydrates, Proteins, Zinc, Molybdenum, Caffeine, Copper, Potassium, Manganese, Chlorides, Bicarbonate and Carbonate, Aluminum, Arsenic, Bromine, Cadmium, Chromium, Sodium, Potassium, Chlorine, Cobalt, Fluorine, Iodine, Citicoline, Tyrosine Phenylalanine, Taurine, Malic Acid, Glucuronolactone, Manganese, Molybdenum Nickel, Phosphorus, Selenium, Silicon, Vanadium, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantetheine, Folic Acid, Vitamin B12, “Unofficial” B Vitamins including Choline and Inositol, Vitamin P (bioflavonoids), and flavoring agents, and/or other vital nutrients, in addition to various homeopathic/alternative substances. In one embodiment the formulation or composition of nutritional supplement matrix comprises 50 to 60 weight % of Chloride, where the chloride is from potassium chloride and sodium chloride; 30 to 40 weight % of Sodium, where the sodium is from sodium chloride; and 5 to 15 weight % of Potassium where the potassium is from potassium chloride.

The gas fraction in the above-mentioned formulation or composition comprises ambient air, oxygen, or nitrogen, wherein the gas fraction maybe in a compressed state. In one embodiment the gas fraction is comprised of oxygen wherein the concentration of oxygen is up to 95% oxygen and can mixed with other gases such as nitrogen.

The enhancer fraction in the above-mentioned formulation or composition is a mucoadhesive enhancer, an absorption enhancer, or a flavoring. Wherein the mucoadhesive enhancer fraction is selected from a group of pectin's or apple pectin's. Wherein the absorption enhancer is selected from a group of glycerin's or vegetable glycerin's. Wherein the flavoring fraction is selected from a group of natural flavoring for foods and artificial flavoring for foods.

The liquid fraction in the above-mentioned formulation or composition is selected from a group of water, distilled water, filtered water, oxygenated water or saline and where the composition is water-soluble. Wherein the amount of water is sufficient to dissolve all elements of the composition and prevent any molecules from precipitating. Furthermore, wherein the liquid fraction can be a combination of liquids and used to adjust the pH of the composition to be close to a physiological pH.

The preservative fraction in the above-mentioned formulation or composition is selected from a group of food preservatives or potassium sorbate. The food preservative fraction helps maintain and extend the shelf life of the composition.

As such, it should be understood that the disclosure is not limited to the particular aspects described herein, but that various changes and modifications may be made without departing from the spirit and scope of this novel concept as defined by the following claims. Further, many other advantages of applicant's disclosure will be apparent to those skilled in the art from the above descriptions and the claims below.

What is claimed is:

1. A system for delivering a composition to an oral mucosa, comprising;
a canister containing pressurized air and at least one liquid composition;
a nozzle housing having a lower opening and an outlet, the lower opening and the outlet are connected by a flow path;
the lower opening of the nozzle housing engaged to the canister to receive an aerosol mixture of the at least one liquid composition from the canister;
the outlet configured to emit the aerosol mixture of the at least one liquid composition; and
the nozzle housing positioning at least one rotating member in or after the flow path.

2. The system for delivering a composition to the oral mucosa according to claim 1, wherein the rotating member is configured to rotate in a lower cavity of the nozzle housing.

3. The system for delivering a composition to the oral mucosa according to claim 1, wherein the rotating member includes multiple blades rotating in a lower cavity the nozzle housing.

4. The system for delivering a composition to the oral mucosa according to claim 1, wherein the rotating member is configured to spin when the pressurized air and the at least one liquid composition are released from the canister.

5. The system for delivering a composition to the oral mucosa according to claim 1, wherein the aerosol mixture of the at least one liquid composition passes through the rotating member.

6. The system for delivering a composition to the oral mucosa according to claim 1, wherein the at least one rotating member is configured to agitate the aerosol mixture of the at least one liquid composition to further atomize the aerosol mixture.

7. The system for delivering a composition to the oral mucosa according to claim 1, wherein a support member extends across a width of an interior of the nozzle housing, and rotating member is rotatably engaged to the support member.

8. The system for delivering a composition to the oral mucosa according to claim 1, wherein an interior of the nozzle housing includes an upper cavity over a lower cavity, the upper cavity has a smaller internal diameter than an internal diameter of the lower cavity, and the rotating member is located in the upper cavity.

9. The system for delivering a composition to the oral mucosa according to claim 1, wherein an interior of the nozzle housing includes an upper cavity over a lower cavity, and the interior narrows from the lower cavity to the upper cavity, and the rotating member is located in the upper cavity.

10. The system for delivering a composition to the oral mucosa according to claim 1, wherein a tubular member is positioned over the outlet, and the rotating member is positioned in the outlet.

11. The system for delivering a composition to the oral mucosa according to claim 1, wherein the aerosol mixture passes through the rotating member after the aerosol mixture is emitted from the outlet.

12. The system for delivering a composition to the oral mucosa according to claim 1, wherein the aerosol mixture passes through the rotating member before the aerosol mixture is emitted from the outlet.

13. The system for delivering a composition to the oral mucosa according to claim 1, wherein a direction of rotation of the rotating member is generally perpendicular to a flow-path of the aerosol mixture.

14. The system for delivering a composition to the oral mucosa according to claim 1, wherein the rotating member includes two separate fans.

15. The system for delivering a composition to the oral mucosa according to claim 1, wherein the rotating member is positioned adjacent to the lower opening of the nozzle housing.

16. The system for delivering a composition to the oral mucosa according to claim 1, wherein the rotating member is positioned between the lower opening and the outlet.

17. A nozzle housing for emitting pressurized air and at least one liquid composition, the nozzle housing comprising:
a lower opening and an outlet, the lower opening and the outlet connected by a flow path;
the lower opening of the nozzle housing configured to engage to a supply of a pressurized air and at least one liquid composition;
the outlet configured to emit an aerosol mixture of the at least one liquid composition;
the nozzle housing positioning at least one rotating member in or after the flow path; and
the at least one rotating member configured to agitate the aerosol mixture of the at least of the at least one liquid composition.

18. A method of delivering an aerosol mixture of oxygen and at least one liquid composition, comprising:
providing a nozzle housing having a lower opening and an outlet, the lower opening and the outlet connected by a flow path;
directing an aerosol mixture of pressurized gas and at least one liquid composition through the flow path of the nozzle housing;
positioning at least one rotating member in or after the flow path;
causing the rotating member to rotate by the aerosol mixture;
agitating the aerosol mixture; and,
delivering the aerosol mixture to an oral mucosa.

19. The method of delivering an aerosol mixture of oxygen and at least one liquid composition according to claim 18, further comprising preventing agglomeration of the at least one liquid composition.

20. The method of delivering an aerosol mixture of oxygen and at least one liquid composition according to claim 18, further comprising engaging the nozzle housing to a supply of pressurized gas and at least one liquid composition.
